(19) United States
(12) Patent Application Publication
(10) Pub. No.: US 2021/0353203 A1
(43) Pub. Date: Nov. 18, 2021

(54) DIAGNOSTICS FOR DETECTION OF ISCHEMIC HEART DISEASE

(71) Applicant: RCE Technologies, Inc., Bermuda Dunes, CA (US)

(72) Inventors: Atandra Burman, Bermuda Dunes, CA (US); Jitto Titus, Acworth, GA (US); Siddharth Biswal, Atlanta, GA (US)

(21) Appl. No.: 17/319,169

(22) Filed: May 13, 2021

Related U.S. Application Data
(60) Provisional application No. 63/024,457, filed on May 13, 2020.

(51) Int. Cl. A61B 5/341; A61B 5/28; A61B 5/0245; A61B 5/024; A61B 5/00; G16H 10/60

(52) U.S. Cl. CPC A61B 5/341; A61B 5/28; A61B 5/0245; A61B 5/02405; G16H 10/60; A61B 5/7275; A61B 5/747; A61B 5/746; A61B 5/7264

(57) ABSTRACT

Aspects of the invention include a computer-implemented method that includes generating an intermediate ECG vector representation and an intermediate optical sensor vector representation. The intermediate ECG vector representation and the intermediate optical sensor vector representation is translated to a joint representation in a vector space. The similarities detected between the electrocardiogram (ECG) data and optical sensor data from the joint vector space representation. Features that are indicative of an ischemic disease of a patient are extracted from the joint vector space representation based at least in part on the detected similarities. The ischemic disease of the patient is detected based at least in part on the extracted features.

### BACKGROUND

The present disclosure generally relates to technical solutions using programmable computing systems, and more specifically, to programmable computing systems for the detection of ischemic heart disease.

The health care system includes a variety of participants, including doctors, nurses, and other hospital-related personnel that provide care for patients. The participants in the health care system utilize a process of risk assessment to identify and analyze patient risks and improve the quality of patient care. Software engineers have developed a variety of tools to evaluate a patient's condition and generate clinical risk scores to help the participants navigate risk assessment. Although these risk assessment tools help the health care system monitor patients, improvements of the tools would be welcomed by the industry.

### SUMMARY

Embodiments of the present invention are directed to diagnostics for the detection of ischemic disease including an acute myocardial injury. A non-limiting example of a computer-implemented method includes generating an intermediate ECG vector representation and an intermediate optical sensor vector representation. The intermediate ECG vector representation and the intermediate optical sensor vector representation are translated to a joint representation in a vector space. The similarities detected between the electrocardiogram (ECG) data and optical sensor data from the joint vector space representation. Features that are indicative of an ischemic disease of a patient are extracted from the joint vector space representation based at least in part on the detected similarities. The ischemic disease of the patient is detected based at least in part on the extracted features.

Other embodiments of the present invention implement features of the above-described method in computer systems and computer program products.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 illustrates a block diagram of components of a system for the detection of ischemic disease in accordance with one or more embodiments of the present invention;

FIG. 2A illustrates an exemplary detection unit for detection of ischemic disease in accordance with one or more embodiments of the present invention;

FIG. 2B illustrates an exemplary narrative unit for detection of ischemic disease in accordance with one or more embodiments of the present invention;

FIG. 3 illustrates a flow diagram of a process for the detection of ischemic disease in accordance with one or more embodiments of the present invention; and

FIG. 4 illustrates a block diagram of a computer system for use in implementing one or more embodiments of the present invention.

The diagrams depicted herein are illustrative. There can be many variations to the diagrams or the operations described therein without departing from the spirit of the invention. For instance, the actions can be performed in a differing order, or actions can be added, deleted, or modified. Also, the term “coupled” and variations thereof describes having a communications path between two elements and does not imply a direct connection between the elements with no intervening elements/connections between them. All of these variations are considered a part of the specification.

### DETAILED DESCRIPTION

One or more embodiments of the present invention provide computer-implemented methods, computing systems, and computer program products for monitoring a patient for the detection of the early signs of ischemic heart disease. The detection of a cardiac injury is relayed to a health care provider to be used for informed clinical decision making, prioritizing resource allocation, and timely intervention.

Coronary Artery Disease (CAD) is characterized by plaque formation in the coronary arteries and is a leading cause for death in men and women around the world. Myocardial ischemia, a progressive state of this disease, is characterized by prolonged blockage from partial or full occlusion of coronary vessels, leading to a downstream oxygen and nutrient deprivation of the muscles and nerve bundles. This results in a myocardial injury with early onset phases of reversible cardiac tissue changes, followed by irreversible cell death, also known as acute myocardial infarction (AMI). Modifiers, such as diabetics susceptible to peripheral neuropathy and women who generally have a higher threshold for pain, may have a proclivity towards silent AMIs that occur in 1 out of 5 AMIs. The precursor silent ischemia in such patients remains asymptomatic and unrecognized for variable periods of time. Additionally, damage in nerve conduction may cause various types of arrhythmias, which in turn can cause sudden cardiac arrest or atrial fibrillations that can potentially lead to neurodegenerative outcomes via cardioembolic strokes. Damage to the myocardium itself can lead to contractile dysfunction of the heart muscle leading to heart failure and possible heart valve defects as well. Myocardial stress and injury can also be identified in patients with heart failure secondary to high blood pressure, infection, or inflammatory response. Related methods of cardiac assessment in patients presenting with chest pain provide a care workflow in a clinical setting, they are not utilized for asymptomatic patients or convenient for remote patient monitoring.

One or more embodiments of the present invention address one or more of the above-described technical shortcomings by providing technical solutions that include computer implemented methods, computing systems and computer program products implantable via a multi-modal convolutional neural network (CNN) that is pre-trained using contrastive loss of ECG data and optical data. A trained CNN is operable for receiving raw ECG data from the back of a patient's body and optical sensor data as a combined representation for analysis and detection of ischemic heart disease. The computer implemented methods, computing systems and computer program products are further operable for receiving stress-related data, activity data, and past medical history data to detect, in real-time, the various states of ischemic disease for asymptomatic patients, including those located outside a clinical setting.

The technical solutions described herein, accordingly address the technical challenges of using a combination of real-time ECG data and optical sensor data for the detection of various states of ischemic disease in asymptomatic patients or convenient for remote patient monitoring. The technical solutions described herein, thus, provide a practical application of a diagnostic system that receives data from an ECG sensor and a non-invasive optical sensor to actively monitor an asymptomatic patient for the early onset of various states of ischemic disease. Further, the technical solutions described here improve existing computing systems and other technical solutions that generate patient diagnostics. The improvements include methods for combining (i) ECG data from the back of the human body affording a stable and robust data source devoid of physical cofounders that pose electrode standoff issues in effective 12 channel ECG data acquisition from varying body morphologies (e.g., obese population with larger abdomens to torso proportions, women with electrodes requiring contact around the breasts) and (ii) optical sensor data representing a unique spectrum of cardiac biomarkers, reduction of computation complexity, preservation of computing resources, and real time monitoring of biometrics of remote patients.

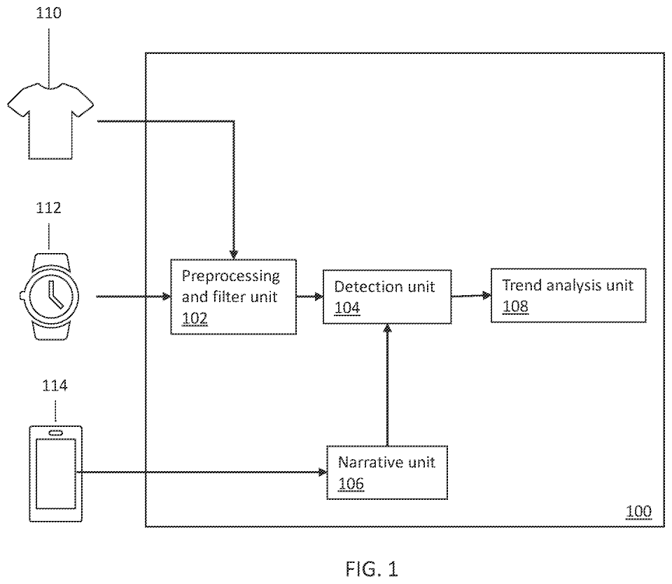

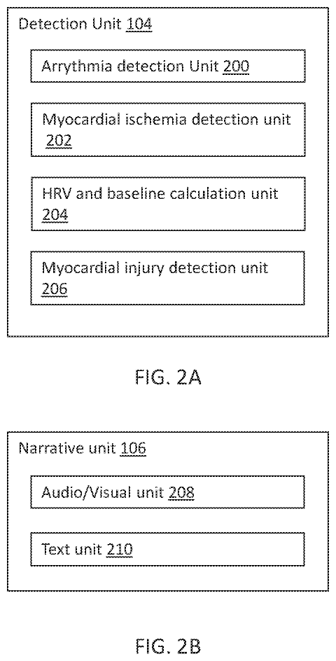

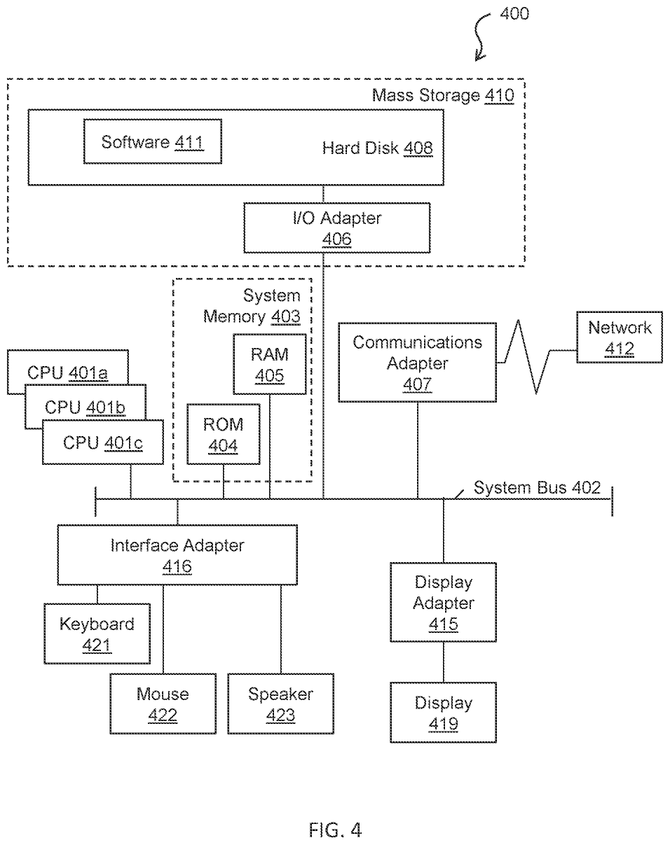

Referring to FIG. 1, a system 100 for the detection of various states of ischemic disease is generally shown in accordance with one or more embodiments of the present invention. The system 100 includes a preprocessing and filter unit 102 for receiving raw data and processing the data for input into a neural network. The system 100 further includes a detection unit 104 for receiving the processed data and detecting an indication of various states of ischemic disease. The system 100 further includes a narrative unit 106 for receiving audio, visual, and textual data and parsing the data to retrieve information indicative of various states of ischemic disease. Exemplary embodiments of the detection unit 104 and the narrative unit 106 are described in further detail with reference to FIGS. 2A and 2B. The system 100 further includes a trend analysis unit 108 for receiving data from the detection unit 104 and the narrative unit 106 and determining whether a patient is at risk of various states of ischemic disease. It should be appreciated that all of the functionality of the system 100 can be performed by a computer system such as the processing system 400 of FIG. 4. Furthermore, the processing system 400 can be implemented in a cloud computing node.

The preprocessing and filter unit 102 is operable to receive data, including sets of time-series data, from multiple sources and standardize the data from each source. For example, the preprocessing and filter unit 102 can import time-series data, collected from ECG device sensors and optical sensors, stored in a server database, and normalize the data to a scale. The normalization allows the trend analysis unit 108 to compare biometric data of the patient to biometric data of another patient. The normalization also permits the trend analysis unit 108 to compare the relative weight of one biometric parameter of a patient to another biometric parameter of the patient. For example, the preprocessing and filter unit 102 can normalize blood pressure data measured in millimeters of mercury (mmHg) and heartbeat data measured in beats per minute (BPM) for future analysis by the trend analysis unit 108.

The preprocessing and filter unit 102 also imports data from the server database and converts the data to a single date time format, such that a collection time of data from one source can be compared with a collection time of data from another source. For example, the preprocessing and filter unit 102 can receive time-series data from ECG device sensors and optical sensors and convert, in parallel, any time values into a single date time format. In some embodiments of the present invention, the ECG sensors are in the form of ten ECG electrodes superimposed from conventional locations (RA: Placed on the right arm or right below the right clavicle, LA: Placed on the left arm or right below the left clavicle, RL: Placed on the right leg or upper right quadrant, LL: Placed on the left leg or upper left quadrant, V1: Placed in the fourth intercostal space to the right of the sternum, V2: Placed in the fourth intercostal space to the left of the sternum, V3: Placed directly between leads V2 and V4, V4: Placed in the fifth intercostal space in the mid-clavicular line, V5: Placed level with V4 at the left anterior mid-axillary line, V6: Placed level with V5 at the mid-axillary line). The preprocessing and filter unit 102 can receive data from multiple sources having a uniform data date format and organize the data into a data structure, for example, a table, to delineate time intervals such as hours, days, months, and years. In this sense, the trend analysis unit 108 can analyze and compare specific time intervals of a patient's history.

In some embodiments of the present invention, the ECG device sensors are incorporated into a wearable garment, for example, a vest. The wearable garment can include a predetermined number of electrodes operable to collect electrical signals from the body that can be represented graphically for detecting any abnormality in heart function. In some embodiments of the present invention, the electrodes are arranged on the back of a wearable garment, such that the electrodes are proximate to a person's back when worn. The electrical signals collected by the electrodes include waveforms or analog signals. Each electrode is operable to receive a signal, for example, such that the collective of electrodes collects 12 ECG signals. In some embodiments of the present invention, the optical sensors are incorporated into wearable devices or garments, for example, a separate smartwatch or the vest. The optical sensors are operable to collect cardiac injury protein levels representing absorption intensities of cardiac biomarker proteins (e.g., troponin I (cTnl), heart type fatty acid binding protein (H-FABP)).

The preprocessing and filter unit 102 may also filter the data from each source to remove any unnecessary data from the time-series data sets. The preprocessing and filter unit 102 may apply statistical methods to identify outlier data points and remove this data from the time-series data sets. For example, the time-series data sets can be represented as a Gaussian distribution, and the preprocessing and filter unit 102 can remove and data points greater than a threshold deviation from the mean. Additionally, any received data that is indicative of an error or corruption can be deleted from the time-series data.

The detection unit 104 is operable to receive real time time-series data from the preprocessing and filter unit 102 and identify data points that are indicative of various states of ischemic disease. For example, the detection unit 104 can receive electrical heart activity data collected from an ECG sensor and protein level data collected from an optical sensor. In addition to heart activity data and protein level data, the detection unit 104 is operable to receive additional biometric data from a patient. The detection unit 104 can be implemented by a neural network, for example, a convolutional neural network (CNN) and is operable to execute multiple artificial intelligence models that are trained to receive the time-series data from the preprocessing and filter unit 102. The detection unit 104 is pre-trained and generates intermediate vectors for each of the modalities of received data (e.g. ECG data, optical sensor data, textual data). The detection unit 104 is fine-tuned on labeled data containing disease specific labels (e.g., myocardial infarction). This fine-tuning adds a softmax layer to a neural network of the detection unit 104 to produce probability values and is trained using a cross-entropy loss function.

In another embodiment of the present invention, the detection unit 104 is pre-trained used both ECG data and optical data in the form of a single input data matrix. Given both the ECG data and optical datasets are time-series data and include mutual information, the datasets are combined into the single input data matrix. Furthermore, training data can be augmented by adding noise to real data. The neural network of the detection unit 104 is optimized using a contrastive loss function that optimizes to find similarities between real data input matrix and an augmented data matrix. This enables the neural network of the detection unit 104 to learn efficient representation of the multimodal ECG and optical data.

The detection unit 104 receives biometric data from multiple sources and generates patient specific predictions as to biomarkers that are indicative of various states of ischemic disease. Unfortunately, unimodal biometric system measure single biometric parameters and are associated with noisy data, lack of performance, spoofing, etc. Therefore, the detection unit 104 operates as a multimodal biometric classifier that can receive data from multiple sources and perform predictions in regard to several biometric characteristics. For example, the detection unit 104 can receive data and make predictions as to arrhythmia, stable angina, unstable angina, myocardial infarction, left ventricular hypertrophy, bundle branch block, myocarditis, pericarditis, and acute heart failure. Each model of the detection unit 104 is operable to receive time-series data, for example, electrical activity of the heart over time and a protein level over time, as inputs for respective models. The detection unit 104 executes each model and generates a respective prediction for whether a biomarker is indicative of obstructive heart disease, for example, whether any one biomarker or combination of biomarkers is suggestive of a partial block or a block of the bloody supply to the heart.

The system 100 can be implemented for various applications including in commercial spaces and clinical settings. For example, the system 100 can be implemented into a kiosk-type stations, in which the kiosk includes a wearable vest and an optical sensor. The kiosk-type station can further include a chair with an ECG vest embedded into the chair, and an optical sensor included in an armrest of the chair. The kiosk-type station can be located, for example, in a commercial setting such as a pharmacy, supermarket, shopping complex. As the kiosk-type station can be located in a public setting, the memory of the kiosk-type station can be segregated into a permanent memory and temporary memory. The temporary memory includes individual data collected from a person and is deleted upon completion of diagnostics. The permanent memory includes computing instructions and memory for operating the system 100. In some embodiments of the present invention, the system 100 is integrated with a local emergency system, such that in the event that a myocardial infarction is detected, the system 100 contacts a local first responders and provides a location of the patient. The system 100 can further be in operable communication with a patient's health care provider. If, however, no myocardial infarction is detected, a diagnostic follow-up appointment is scheduled with the patient's health care provider's calendaring system.

The system 100, can further be implemented as a portable application, for example, for use in an ambulance or at a hospital bedside. The system 100 can be in operable communication with a wearable garment fitted with ECG sensors and also in communication with a wearable device fitted with optical sensors. In some embodiments, the wearable vest includes the optical sensors. The system 100 can be implemented by a portable computing system that is either physically attached to the wearable vest and optical sensors or is in wireless communication with the wearable vest and optical sensors. For example, the system 100 can communicate with the ECG sensors and the optical sensors via near field communication, Bluetooth, or other appropriate communication protocol.

The detection unit 104 models are trained using a contrastive learning techniques and supervised learning. Contrastive learning enables the detection unit 104 to receive unlabeled ECG data and unlabeled optical data and determine which data are similar to each other and which are different from each other. Contrastive learning optimizes the neural network of the detection unit 104 for a contrastive loss function. A contrastive loss function is defined as a loss function, which can be optimized to find a similarity between vectors (e.g., ECG data vector, optical data vector, and word vector). The detection unit 104 is trained by receiving a vector representation of ECG data and a vector representation of the optical data (and in some embodiments, a vector representation of text data), including real-time ECG data and the optical data. The vector representation of the ECG data can be representative of ECG data from multiple channels of an ECG sensor device. The vector representation of the optical data can representative of optical data from multiple channels of an optical sensor.

The detection unit 104 is then trained to maximize the similarities between the vector representation of ECG data and a vector representation of the optical data by minimizing a contrastive loss function. For example, each of the ECG data and the optical data can pass through a respective encoder of the detection unit 104. The encoder generates a respective intermediate representation of each vector. Each intermediate vector representation is passed through several dense layers (projection head) of the neural network, which transform each vector into a joint representation or latent space. From the joint representation, the detection unit 104 extracts features from the transformed ECG data and the transformed optical data to generate feature vectors. The detection unit 104 then determines a similarity between the two vectors, for example, by using a cosine similarity function. The similarities between the extracted features are further determined by minimizing the aforesaid mentioned contrastive loss function. As the detection unit 104 is trained to recognize the similarities, it learns that the ECG data and the optical data have similarities.

Once the detection unit 104 is trained to recognize the similarities between the ECG data and the optical data, a classification layer of the detection unit 104 is used to train a fine-tuned version of the neural network. The fine tuning can be performed such that the detection unit 104 is trained to associate the combined similarity data with different labels such as myocardial infarction (MI) and non-myocardial infarction (Non-MI). It should be appreciated that although two classes are described, the detection unit 104 is operable to classify greater than two classes.

In some embodiments of the present invention, the classification layer is trained using a cross-entropy loss function. The cross-entropy loss function is employed by the detection unit 104 in conjunction with a Softmax function output of the neural network. Based on the result of the cross-entropy loss function, the hyperparameters (e.g., weights, biases) of the model are adjusted through backpropagation to improve the accuracy of the model's classifier. The detection unit 104 further includes a self-attention layer, which is trained to determine a hierarchy for features used to generate a classification. The self-attention layer allows the detection unit 104 to recognize important features for classification and speed up the classification process.

A gold standard diagnostic is applied to measure the accuracy of the model in relation to previous versions of the model. If the model performs better than a previous model, the previous model is deleted, and the detection unit 104 uses the new model. If the model does not perform better than the previous model, the model is deleted, and the detection unit 104 continues to use the previous model. Once the detection selects a model, the model is pruned to decrease latency and model size. The models can be pruned through unstructured pruning or structured pruning. In unstructured pruning, connections between nodes are removed by setting a weight value to zero and effectively multiplying the output of an activation function by zero. In structured pruning, the channels between nodes are removed entirely. In some embodiments of the present invention, whether or not a model is pruned is based at least in part on whether the system is operating on a central device such a server or an edge device such as a patient's smartphone, laptop, or personal computer. If the model is executed on a server, the training does not include pruning the model. If, however, the model is being executed on an edge device, the training does include pruning to reduce the complexity of the computations and preserve processor space.

The narrative unit 106 is operable for receiving patient or healthcare worker generated data. For example, the narrative unit 106 is operable to receive patient notes, image scans, recorded oral notes, answers to patient questionnaires and the like. An exemplary embodiment of the narrative unit 106 is described in further detail in relation to FIG. 2B below. The narrative further employs a model to analyze any text or image to determine whether any information is indicative of whether the patient has obstructive coronary heart disease.

The trend analysis unit 108 receives an output from the detection unit 104 to assist in determining whether the patient is at risk of ischemic heart disease. In addition to the output of the detection unit 104, the trend analysis unit 108 is operable to receive demographics data, a patient's fitness related data, a patient's family history, and other related data. The trend analysis unit 106 applies the data to generate a risk stratification for the patient. The risk stratification is a quantitative measurement of the patient's probability of developing obstructive coronary artery disease or myocardial infarction. The trend analysis unit 108 further compares the patient's risk stratification to a table for informative actions. For example, if a patient is at a high risk of experiencing a medical emergency, the trend analysis unit 108 can cause an alert to be issued to local medical services or the patient's primary care physician. If the risk stratification suggests a medium risk, the trend analysis unit 108 can raise patient awareness (e.g., via text, or email) and suggest corrective measures for the patient. If the risk stratification suggests a low risk, the trend analysis unit 108 can continue monitoring the patient.

The system 100 is operable to receive patient data from one or multiple sources in real-time. The sources include integrated wearables 110 that include sensors such as a twelve-channel electrocardiogram (ECG) sensor for monitoring electrical signals from the patient's heart. The ECG sensor can be, for example, integrated with a patient's clothing, such that the patient can wear the ECG sensor as clothing and transmit data to the system 100 in real-time. The ECG sensor can be incorporated into a fabric, for example as part of a garment, such as a shirt, pant, underwear, etc. The ECG sensor, additionally or alternatively, is worn separate from the garment, for example as part of a watch, shoes, and the like.

The sources further include wearable devices 112, for example, optical sensors integrated into smart watches, and garments operable to non-invasively monitor a patient's protein levels and detect any progression of obstructive coronary heart disease. The optical sensors can be integrated into a wearable device, such as a smart watch, and be configured to transmit an electromagnetic signal to a patient's epidermis. The optical sensors are further configured to receive a reflected signal indicative of spectral absorption back from the patient. The trend analysis unit 108 analyzes the spectral absorption to determine the presence of specific protein biomarkers in the patient's blood (e.g., troponin I (cTnl), heart type fatty acid binding protein (H-FABP)). The optical sensors can provide narrow filter based acquisition of specific proteins, such as cTnl, h-FABP, or broad filter based acquisition of cardiac inflammatory biomarkers. The trend analysis unit 108 is operable to determine whether the presence and concentration of one or more biomarkers in a patient's blood is indicative of obstructive coronary heart disease. The wearable devices 112 further include devices for collecting additional biometric data (e.g., heart rate, pulse, steps walked, accelerometer based positioning, inter-compartment fluid distribution).

The sources further include mobile and stationary computing devices 114, for example, audio capturing devices, image capturing devices, personal computers, laptops, tables, and the like. The mobile and stationary computing devices 114 are operable to receive user initiated data and transmit the date to the trend analysis unit 108. For example, the sources include a smartphone or laptop computer for receiving audio recordings of a clinician's notes, a scanning device for receiving a patient's x-ray images, a computed tomography device, a mobile or stationary computing device for receiving demographics data.

The sources are operable to communicate with the system 100 using technologies such as Ethernet, fiber optics, microwave, xDSL (Digital Subscriber Line), Wireless Local Area Network (WLAN) technology, Near Field Communication (NFC) wireless cellular technology, BLUETOOTH technology and/or any other appropriate technology

Referring to FIG. 2A, an exemplary embodiment of the detection unit 104 is shown. The exemplary detection unit 104 includes an arrhythmia detection unit 200, a myocardial ischemia unit 202, an HRV and baseline calculation unit 204, and a myocardial injury detection unit 206. The arrhythmia detection unit 200 receives ECG data in the form of time-series data from a patient and classifies the patient's heart rhythm as ranging from normal or abnormal. For example, the arrhythmia detection unit 200 employs a deep neural network (e.g., a one-dimensional convolutional neural network) for feature extraction and classification. The arrhythmia detection unit 200 receives a waveform from an ECG sensor and converts the waveform to an intermediate representation of the waveform The detection unit 104 later extracts features from a joint representation that includes the arrythmia information and applies a model to select a classification from a set of classifications ranging from a normal to an abnormal heart rhythm.

The myocardial ischemia unit 202 is operable to receive patient data, (e.g., blood flow data), and predict whether the patient is at risk of myocardial ischemia. For example, the myocardial ischemia unit 202 can employ an artificial neural network (ANN) to extract features from a joint representation that includes representations of the morphological patterns on ECG, (e.g., features indicative of an ST depression, T-wave inversion, flat T-wave, hyperacute T-wave, new Q wave, or ST elevation), and optical (e.g., differentials of optical device channel outputs) from the data and apply the extracted features to a model. The model analyzes the data both temporally and spatially to output a prediction as to whether the data suggests the patient is at risk for myocardial ischemia.

The HRV and baseline calculation unit 204 is operable to measure a variation in time between each heartbeat and is indicative of stress in a patient's life. The HRV and baseline calculation unit 204 receives the ECG data and calculates the HRV for the patient. The HRV and baseline calculation unit 204 further receives ECG data and calculates a baseline heart rate of the patient based on the ECG data. The HRV and baseline calculation unit 204 further generates a baseline of a patient's normal levels of cardiac biomarkers, such as Troponins, H-FABP, NT-ProBNP, CK-MB, CRP, and suPar. The information can be utilized by a classification layer of the detection unit 104 to output a prediction as to whether the patient is at risk.

The myocardial injury detection unit 206 is operable to receive an output from the arrhythmia detection unit 200, the myocardial ischemia unit 202, and the HRV and baseline calculation unit 204, and make a prediction as to whether a patient exhibits a risk of obstructive coronary heart disease. In some embodiments of the present invention, the myocardial injury detection unit 206 is implemented as a classification layer of a neural network of the detection unit 104. The myocardial injury detection unit 206 can make a prediction as to whether the patient exhibits a risk of ischemic heart disease.

Referring to FIG. 2B, an exemplary embodiment of the narrative unit 106, is shown. The narrative unit 106 includes audio/visual unit 210 and a text unit 212. The audio/visual unit 210 is operable to receive an image (e.g., x-ray image, CT scan image, point cloud image) and extract domain-specific features describing objects in the image. The audio/visual unit 210 can further employ a computer vision model to detect and label domain specific-objects in the image. The audio/visual unit 210 can further label each object class in the image. The audio/visual unit 210 can represent the extracted features and identified object classes in a joint representation.

The audio/visual unit 210 is further operable to receive audio data (e.g., health care worker's audio notes, patient's audio report, electronic medical records) and extract domain-specific audio features from audio waveforms. The audio/visual unit 210 can further employ an audio analysis model to detect and label domain specific speech in the audio waveform. The audio/visual unit 210 can further label text in the waveform. The audio/visual unit 210 can represent the extracted features and identified object classes in a joint representation.

The text unit 212 is operable to receive textual information in electronic format (e.g., clinician's notes, lab reports). The text unit 212 can apply a model that uses natural language processing (NLP) techniques to analyze the text and determine whether the text relates to whether it is more likely or not that a patient has obstructive coronary heart disease. The model can be, for example, a word embedding model. The text unit 212 can employ various techniques to derive a context of the text. The text unit 212 can organize the text into a parse tree to assist in determining the context. The text unit 212 can parse the text through various methods, for example, a constituency parsing method. A constituency parsing method involves reconstructing a text into a constituency-based parse tree describing the passage's syntactic structure based on a phase structure grammar. Phase structure grammar is based upon constituency relations between tokens as opposed to dependency relations between tokens. The text unit 212 can also employ a dependency parsing method, in which a parse tree is constructed based on a dependency relation between tokens. The text unit 212 can rely on the organization of the tokens in the text tree to determine a context of the text. This can be based on words surrounding a target word in the text, or using a target word to derive the meaning of the surrounding words.

The phrases “neural network” and “machine learning” broadly describes a function of electronic systems that learn from data. A machine learning system, engine, or module can include a machine learning algorithm that can be trained, such as in an external cloud environment (e.g., the cloud computing environment 50), to learn functional relations between inputs and outputs that are currently unknown. In one or more embodiments, machine learning functionality can be implemented using a detection unit 104, a narrative unit 106, and a trend analysis unit 108, each having the capability to be trained to perform a currently unknown function. In machine learning and cognitive science, neural networks are a family of statistical learning models inspired by the biological neural networks of animals, and in particular, the brain. Neural networks can be used to estimate or approximate systems and functions that depend on a large number of inputs.

The detection unit 104, narrative unit 106, and trend analysis unit 108 can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in the detection unit 104, narrative unit 106, and trend analysis unit 108 that carry electronic messages between simulated neurons are provided with numeric weights that correspond to the strength or weakness of a given connection. During training, the weights can be adjusted and tuned based on experience, making the detection unit 104, narrative unit 106, and trend analysis unit 108 adaptive to inputs and capable of learning. After being weighted and transformed by a function determined by the network's designer, the activation of these input neurons are then passed to other downstream neurons, which are often referred to as “hidden” neurons. This process is repeated until an output neuron is activated. The activated output neuron determines which character was read.

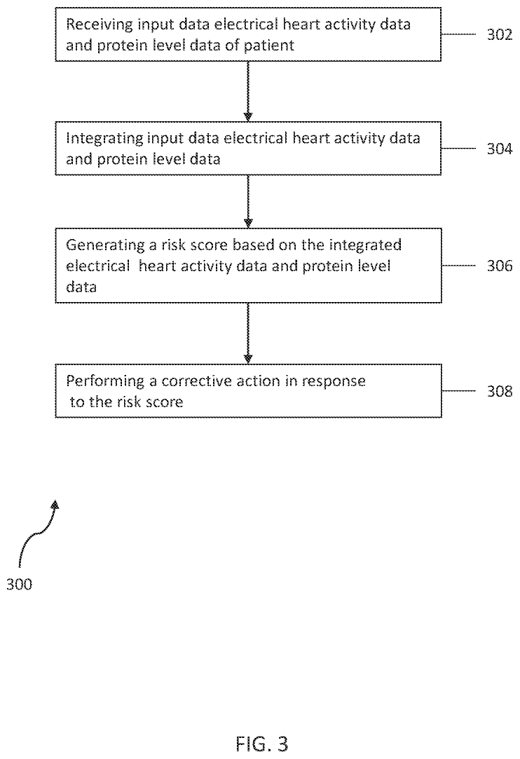

Referring to FIG. 3, a flow diagram 300 of a process for the detection of various states of ischemic disease in accordance with one or more embodiments of the present invention. It should be appreciated that all or a portion of the processing shown in FIG. 3 can be performed by a computer system, such as system 100 of FIG. 1. At block 302, a detection unit 104 receives input data including electrical heart activity data and protein level data of a patient. The electrical heart activity data can be received from a twelve-channel ECG sensor. The ECG sensor can further be integrated into the patient's clothing and worn outside a clinical setting. The protein level data can be received from an optical sensor that emits an electromagnetic wave towards a patient's epidermis and collects a signal reflected from the patient.

At block 304, the detection unit 104 integrates the electrical heart activity data with the protein level data in a joint representation to determine the patient's condition. The detection unit 104 can apply one or more neural networks to analyze the time-series data by extracting features and classifying the data as either indicative or not indicative of ischemic disease. At block 306, a trend analysis unit 108 uses the output of the detection unit 104 to generate a risk stratification for the patient. The trend analysis unit 108 further inputs the risk stratification to a risk stratification model that computes the probability of an ischemic heart event (normal, stable angina, unstable angina, NSTEMI, STEMI) to determine an appropriate informative action.

At block 308, the trend analysis unit 108 causes an appropriate informative action to be taken. For example, if a patient is at a high risk of or experiencing a medical emergency, the trend analysis unit 108 can cause an alert to be issued local medical services or the patient's primary care physician. If the risk stratification suggests a medium risk, the trend analysis unit 108 can cause (e.g., via text or email) suggestive measures for the patient. If the risk stratification suggests a low risk, the trend analysis unit can continue monitoring the patient.

In one or more embodiments of the present invention, the hardware/software modules in the system 100 from FIG. 1 can be implemented on the processing system 400 found in FIG. 4. Turning now to FIG. 4, a computer system 400 is generally shown in accordance with an embodiment. The computer system 400 can be an electronic, computer framework comprising and/or employing any number and combination of computing devices and networks utilizing various communication technologies, as described herein. The computer system 400 can be easily scalable, extensible, and modular, with the ability to change to different services or reconfigure some features independently of others. The computer system 400 may be, for example, a server, desktop computer, laptop computer, tablet computer, or smartphone. In some examples, computer system 400 may be a cloud computing node. Computer system 400 may be described in the general context of computer system executable instructions, such as program modules, being executed by a computer system. Generally, program modules may include routines, programs, objects, components, logic, data structures, and so on that perform particular tasks or implement particular abstract data types. Computer system 400 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, the computer system 400 has one or more central processing units (CPU(s)) 401a, 401b, 401c, etc. (collectively or generically referred to as processor(s) 401). The processors 401 can be a single-core processor, multi-core processor, computing cluster, or any number of other configurations. The processors 401, also referred to as processing circuits, are coupled via a system bus 402 to a system memory 403 and various other components. The system memory 403 can include a read only memory (ROM) 404 and a random access memory (RAM) 405. The ROM 404 is coupled to the system bus 402 and may include a basic input/output system (BIOS), which controls certain basic functions of the computer system 400. The RAM is read-write memory coupled to the system bus 402 for use by the processors 401. The system memory 403 provides temporary memory space for operations of said instructions during operation. The system memory 403 can include random access memory (RAM), read only memory, flash memory, or any other suitable memory systems.

The computer system 400 comprises an input/output (I/O) adapter 406 and a communications adapter 407 coupled to the system bus 402. The I/O adapter 406 may be a small computer system interface (SCSI) adapter that communicates with a hard disk 408 and/or any other similar component. The I/O adapter 406 and the hard disk 408 are collectively referred to herein as a mass storage 410.

Software 411 for execution on the computer system 400 may be stored in the mass storage 410. The mass storage 410 is an example of a tangible storage medium readable by the processors 401, where the software 411 is stored as instructions for execution by the processors 401 to cause the computer system 400 to operate, such as is described herein below with respect to the various Figures. Examples of computer program products and the execution of such instruction is discussed herein in more detail. The communications adapter 407 interconnects the system bus 402 with a network 412, which may be an outside network, enabling the computer system 400 to communicate with other such systems. In one embodiment, a portion of the system memory 403 and the mass storage 410 collectively store an operating system, which may be any appropriate operating system to coordinate the functions of the various components shown in FIG. 4.

Additional input/output devices are shown as connected to the system bus 402 via a display adapter 415 and an interface adapter 416 and. In one embodiment, the adapters 406, 407, 415, and 416 may be connected to one or more I/O buses that are connected to the system bus 402 via an intermediate bus bridge (not shown). A display 419 (e.g., a screen or a display monitor) is connected to the system bus 402 by a display adapter 415, which may include a graphics controller to improve the performance of graphics intensive applications and a video controller. A keyboard 421, a mouse 422, a speaker 423, etc. can be interconnected to the system bus 402 via the interface adapter 416, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 4, the computer system 400 includes processing capability in the form of the processors 401, and, storage capability including the system memory 403 and the mass storage 410, input means such as the keyboard 421 and the mouse 422, and output capability including the speaker 423 and the display 419.

In some embodiments, the communications adapter 407 can transmit data using any suitable interface or protocol, such as the internet small computer system interface, among others. The network 412 may be a cellular network, a radio network, a wide area network (WAN), a local area network (LAN), or the Internet, among others. An external computing device may connect to the computer system 400 through the network 412. In some examples, an external computing device may be an external webserver or a cloud computing node.

It is to be understood that the block diagram of FIG. 4 is not intended to indicate that the computer system 400 is to include all of the components shown in FIG. 4. Rather, the computer system 400 can include any appropriate fewer or additional components not illustrated in FIG. 4 (e.g., additional memory components, embedded controllers, modules, additional network interfaces, etc.). Further, the embodiments described herein with respect to computer system 400 may be implemented with any appropriate logic, wherein the logic, as referred to herein, can include any suitable hardware (e.g., a processor, an embedded controller, or an application specific integrated circuit, among others), software (e.g., an application, among others), firmware, or any suitable combination of hardware, software, and firmware, in various embodiments.

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

One or more of the methods described herein can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

In some embodiments, various functions or acts can take place at a given location and/or in connection with the operation of one or more apparatuses or systems. In some embodiments, a portion of a given function or act can be performed at a first device or location, and the remainder of the function or act can be performed at one or more additional devices or locations.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The present disclosure has been presented for purposes of illustration and description, but is not intended to be exhaustive or limited to the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

The diagrams depicted herein are illustrative. There can be many variations to the diagram or the steps (or operations) described therein without departing from the spirit of the disclosure. For instance, the actions can be performed in a differing order or actions can be added, deleted or modified. Also, the term “coupled” describes having a signal path between two elements and does not imply a direct connection between the elements with no intervening elements/connections therebetween. All of these variations are considered a part of the present disclosure.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
generating, by a processor, an intermediate ECG vector representation and an intermediate optical sensor vector representation;
translating, by the processor, the intermediate ECG vector representation and the intermediate optical sensor vector representation to a joint representation in a vector space;
detecting, by the processor, similarities detected between the electrocardiogram (ECG) data and optical sensor data from the joint vector space representation; and
extracting, by the processor, features indicative of an ischemic disease of a patient from the joint vector space representation based at least in part on the detected similarities; and
detecting, by the processor, the ischemic disease of the patient based at least in part on the extracted features.

2. The computer-implemented method of claim 1, wherein the intermediate ECG vector representation is based on data received from a setup of ten ECG electrodes on the back of the body superimposed from LA, RA, LL, RL, V1, V2, V3, V4, V5, and V6 locations and integrated into the clothing of the patient.

3. The computer-implemented method of claim 1, wherein the optical sensor data representation comprises cardiac injury biomarker data of the patient.

4. The computer-implemented method of claim 1 further comprising
receiving, by the processor, optical sensor data from the patient;
determining cardiac injury protein levels of the patient based at least in part on spectral absorption detected from the reflected wave; and
generating the intermediate optical sensor vector representation based at least in part on the determined cardiac injury protein levels.

5. The computer-implemented method of claim 1, wherein the ECG data comprises at least one of arrhythmia data, myocardial data, and heart rate variability.

6. The computer-implemented method of claim 1 further comprising:
receiving electronic medical records of the patient;
applying the ECG data, the cardiac injury protein levels, and the electronic medical records as inputs into one or more neural networks;
receiving, from the one or more neural networks, an output predicting whether patient exhibits an indication of ischemic disease.

7. The computer-implemented method of claim 1, wherein the informative actions comprise alerting an emergency medical system, transmitting corrective suggestions to the patient; and continue monitoring the patient.

8. A system comprising:
a memory having computer readable instructions; and
one or more processors for executing the computer readable instructions, the computer readable instructions controlling the one or more processors to perform operations comprising:
generating an intermediate ECG vector representation and an intermediate optical sensor vector representation;
translating the intermediate ECG vector representation and the intermediate optical sensor vector representation to a joint representation in a vector space;
detecting similarities detected between the electrocardiogram (ECG) data and optical sensor data from the joint vector space representation; and
extracting features indicative of an ischemic disease of a patient based at least in part on the detected similarities; and
detecting the ischemic disease of the patient from the joint vector space representation based at least in part on the extracted features.

9. The system of claim 8, wherein the intermediate ECG vector representation is based on data received from a setup of ten ECG electrodes on the back of the body superimposed from LA, RA, LL, RL, V1, V2, V3, V4, V5, and V6 locations and integrated into the clothing of the patient.

10. The system of claim 8, wherein the optical sensor data representation comprises cardiac injury biomarker data of the patient.

11. The system of claim 8, the operations further comprising:
receiving optical sensor data from the patient;
determining cardiac injury protein levels of the patient based at least in part on spectral absorption detected from the reflected wave; and
generating the intermediate optical sensor vector representation based at least in part on the determined cardiac injury protein levels.

12. The system of claim 8, wherein the ECG data comprises at least one of arrhythmia data, myocardial data, and heart rate variability.

13. The system of claim 8, the operations further comprising:
receiving electronic medical records of the patient;
applying the ECG data, the cardiac injury protein levels, and the electronic medical records as inputs into one or more neural networks;
receiving, from the one or more neural networks, an output predicting whether patient exhibits an indication of ischemic disease.

14. The system of claim 8, wherein the informative actions comprise alerting an emergency medical system, transmitting corrective suggestions to the patient; and
continue monitoring the patient.

15. A computer program product comprising a computer readable storage medium having program instructions embodied therewith, the program instructions executable by a processor to cause the processor to perform operations comprising:
generating an intermediate ECG vector representation and an intermediate optical sensor vector representation;
translating the intermediate ECG vector representation and the intermediate optical sensor vector representation to a joint representation in a vector space;
detecting similarities detected between the electrocardiogram (ECG) data and optical sensor data from the joint vector space representation; and
extracting features indicative of an ischemic disease of a patient from the joint vector space representation based at least in part on the detected similarities; and
detecting the ischemic disease of the patient based at least in part on the extracted features.

16. The computer program product of claim 15, wherein the intermediate ECG vector representation is based on data received from a setup of ten ECG electrodes on the back of the body superimposed from LA, RA, LL, RL, V1, V2, V3, V4, V5, and V6 locations and integrated into the clothing of the patient.

17. The computer program product of claim 15 wherein the optical sensor data representation comprises cardiac injury biomarker data of the patient.

18. The computer program product of claim 15, the operations further comprising:
receiving, by the processor, optical sensor data from the patient;
determining cardiac injury protein levels of the patient based at least in part on spectral absorption detected from the reflected wave; and
generating the intermediate optical sensor vector representation based at least in part on the determined cardiac injury protein levels.

19. The computer program product of claim 15, wherein the ECG data comprises at least one of arrhythmia data, myocardial data, and heart rate variability.

20. The computer program product of claim 15, the operations further comprising:
receiving electronic medical records of the patient;
applying the ECG data, the cardiac injury protein levels, and the electronic medical records as inputs into one or more neural networks;
receiving, from the one or more neural networks, an output predicting whether patient exhibits an indication of ischemic disease.
